(19) United States
(12) Patent Application Publication
(10) Pub. No.: US 2001/0051179 A1
(43) Pub. Date: Dec. 13, 2001

(54) METHOD FOR PREVENTING THE SPREAD OF INFECTIOUS MICROORGANISMS USING CYANOACRYLATE

(76) Inventor: CHARLES L. BERMAN, NEW YORK, NY (US)

Correspondence Address: STEPHEN E FELDMAN, 12 EAST 41ST STREET, SUITE 1302, NEW YORK, NY, 10017

(21) Appl. No.: 08/816,763

(22) Filed: Mar. 14, 1997

Related U.S. Application Data
(63) Continuation of application No. 08/717,240, filed on Sep. 20, 1996, now abandoned.

(51) Int. Cl. A61K 31/44

(52) U.S. Cl. 424/445

(57) ABSTRACT

There is disclosed a method for preventing infectious microorganisms, especially herpes, from forming an open sore, and if a sore forms, from spreading from open sores or wounds by applying cyanoacrylate directly to such an open sore or wound in an amount sufficient to cover and seal the open wound or sore until it is healed.

This is a continuation-in-part of the application entitled METHOD FOR PREVENTING THE SPREAD OF INFECTIOUS MICROORGANISMS USING N-BUTYL CYANOACRYLATE by Charles Berman, Ser. No. 08/717,240, filed on Sep. 20, 1996.

### BACKGROUND OF THE INVENTION

This invention relates to a method for preventing the spread of microorganisms that migrate from within one's body to and through one's skin where they appear as open sores or wounds. By applying cyanoacrylate directly to such open sores or wounds, the infectious microorganisms that cause them are prevented from spreading, becoming worse, or being transmitted to other persons and objects.

Both mono-and polymeric cyanoacrylate have been used to treat various skin disorders and as surgical adhesives. For example, U.S. Pat. No. 3,667,472 to Halpern discloses the use of C2-C4 alkyl alpha-cyanoacrylate, including butyl-cyanoacrylate, as surgical adhesives so that when these adhesives are applied to moist, living tissue, polymerization takes place in situ to firmly bond the tissue together.

Autopolymerizable alpha-cyanoacrylate surgical adhesive compositions containing a dye are disclosed in U.S. Pat. No. 3,699,076 to Thomsen, et. al. The inclusion of a dye or coloring material makes the adhesive readily visible and prevents premature polymerization of the adhesive composition. The compositions can contain alkyl cyanoacrylate such as butyl-cyanoacrylate.

In U.S. Pat. No. 4,752,472 to Kligman, alpha-cyanoacrylate is disclosed as the polymerizable adhesive used to cosmetically remove materials from the surface and the sebaceous follicles of human skin.

U.S. Pat. No. 5,403,591 to Tighe, et. al. discloses the use of a cyanoacrylate adhesive which is applied to the surfaces of the skin that are prone to ulceration in order to prevent skin ulcers from forming. The adhesive is applied to an area of the skin that is not contiguous with a formed, open pressure sore.

### DESCRIPTION OF THE INVENTION

This invention is directed to a method for preventing the spread of infectious microorganisms that originate within one's body and migrate to the surface of the skin where they erupt into and appear as open sores or wounds. These open sores or wounds typically excrete a clear liquid containing the infectious microorganisms which can serve as the carrier to spread the infectious microorganisms to various parts of one's body, from one person to another, and to other host objects such as foods, dinner ware, eating utensils, and the like. It has now been found that by applying a coating of cyanoacrylate directly to these open sores or wounds in an amount sufficient to cover and seal the open sores or wounds, the infectious microorganisms are contained and prevented from spreading until healing is completed without endangering one's self, other persons, or other host objects. In a similar manner, applying the cyanoacrylate directly to the prodrome of the sore will prevent the open sore from forming.

Typical and illustrative of an infectious microorganism that erupts into an open sore or wound and that can easily and readily be spread over one's body or be transmitted to other persons or host objects is the Herpes Simplex virus. This virus sheds through the skin in small vesicles that form dendrites or lesions. A clear liquid carrying the virus exudes through the vesicles and the area surrounding the dendrites where there are no vesicles also becomes infected as the virus spreads through the skin.

Normally, the Herpes Simplex virus resides in the Dorsal Cort ganglia. When evidenced as a lip “cold sore”, the virus lives in the ganglia of the fifth (V) cranial nerve. When placed under stress or for other reasons, the virus migrates along the nerve to the skin where it first indicates a peridonal sign in the form of a burning sensation (prodrome) or at times, a numbness of the lips, usually at the mucocutaneous junction. Subsequently, swelling and redness develop, followed by the formation of papillae or clusters of papulla which combine to form vesicles. Eventually, these vesicles rupture to release a clear fluid containing the virus. If left untreated, these vesicles will become encrusted and healing will normally follow in about 10 days.

One having an open sore or wound excreting clear liquid containing the Herpes Simplex virus can readily spread the virus to other parts of the body such as by touching the liquid with one's hands and then touching another part of one's body. In this way, the virus can be transferred to one's eyes where it can damage the cornea and lens and result in blindness if not treated early. In the same manner, one can transmit the virus to another person and other host objects.

When a coating of cyanoacrylate is applied directly onto the open vesicles and permitted to dry or “cure”; i.e. polymerize, a plastic bandage forms over the open vesicles keeping the virus in. Not only is the virus contained and prevented from spreading, but any accompanying pain is immediately reduced and drainage of the clear liquid is immediately stopped. Treating the open vesicles twice a day in this manner is continued until the sore or wound is healed; i.e. closes, typically in about three to four days.

The quantity of cyanoacrylate that should be applied to treat an open wound or sore to prevent the spread of infectious microorganisms is that amount that will be sufficient to completely cover the open sore or wound. Typically, the quantity of cyanoacrylate applied should be sufficient to form a film over the open sore or wound of from about 0.01 mm. to about 0.5 mm. thick, preferably from about 0.05 mm. to about 0.3 mm. thick. In addition, the amount of cyanoacrylate applied should also extend to cover the peripheral area surrounding the open sore or wound to form an extra safety shield. This peripheral area can typically extend outwardly from the open sore or wound a distance of from about 0.05 cm. to about 0.5 cm., preferably from about 0.1 cm. to about 0.3 cm.

Any conventional, commercially available means can be employed to apply the cyanoacrylate to an open sore or wound provided such means will not adhere to the open sore or wound or to the peripheral skin area immediately surrounding the open sore or wound. Such means should also prevent the person applying the cyanoacrylate from coming into contact with the open sore or wound or from becoming adhered either to the applicator means, or the open sore or wound, or the skin area immediately surrounding the open sore or wound. Illustrative, commercially available means that can be employed are applicators such as sprays, containers that deliver in drops, brushes, medical swabs, paddles, and the like. Whenever an applicator is used that requires the applicator means to come into contact with the open sore or wound, the applicator should be one that can then be discarded and not re-used.

Before the open Herpes sore forms, an area where the sore will form becomes sensitive and itchy, and is known as a prodrome. The application of cyanoacrylate to the prodrome prevents the sore from forming. The cyanoacrylate can also be applied in the same manner as previously described to the prodrome.

The procedure can be used to treat other Herpes infections, including genital herpes and chicken pox.

The cyanoacrylates that can be used to practice the method of the invention are those that are of the class consisting of butyl-cyanoacrylate, methyl-cyanoacrylate, allyl-cyanoacrylate, iso-butyl-cyanoacrylate, n-butyl-cyanoacrylate, ethyl cyanoacrylate, or octyl-cyanoacrylate.

The invention is further illustrated through the following cases which are set forth to further exemplify the invention.

### EXAMPLE 1

A Caucasian male, age 69, had open sores on his lower lip from which a clear liquid was exuding. The sores were diagnosed as resulting from the Herpes Simplex virus. His initial office treatment consisted of swabbing the open sores with n-butyl-cyanoacrylate using a surgical swab until the sores and surrounding tissue were completely covered. He was given a supply of n-butyl-cyanoacrylate and surgical paddles for self-application and instructed on their use. He was cautioned not to close his lips or press them together after applying the n-butyl-cyanoacrylate for two to three minutes so that the coating over the sores would be completely dry. His twice daily treatment was continued for three days at which time the sores were no longer exudating.

### EXAMPLE 2

An individual had open sores on his lip from which a clear liquid was exuding. The sores were diagnosed as Herpes Simplex virus. He was instructed to coat the sores with ethyl cyanoacrylate (C₆H₇NO₂) in a similar manner to Example 1. A twice-daily treatment was continued for several days at which time the sores were no longer exudating.

### EXAMPLE 3

An individual had open sores on his lip from which a clear liquid was exuding. The sores were diagnosed as resulting from the Herpes Simplex virus. He was instructed to coat the sores with 2 acetyl cyanoacrylate using a surgical swab until the sores and surrounding tissue were completely covered. His twice-daily treatment was continued for three days at which time the sores were no longer exuding.

### EXAMPLE 4

A Caucasian male, age 69, had itching on his lower lip. The itching was diagnosed as resulting from a prodrome of the Herpes Simplex virus. His initial office treatment consisted of being told to swab the prodrome with n-butyl-cyanoacrylate using a surgical swab until the prodrome and surrounding tissue were completely covered. He was given a supply of n-butyl-cyanoacrylate and surgical paddles for self-application and instructed on their use. His twice-daily treatment was continued for three days at which time the prodrome disappeared and no sores formed or exuded.

### EXAMPLE 5

An individual had open sores on his lip from which a clear liquid was exuding. The sores were diagnosed as resulting from the Herpes Simplex virus. He was instructed to coat the sores with octyl-cyanoacrylate using a surgical swab until the sores and surrounding tissue were completely covered. His twice-daily treatment was continued for three days at which time the sores were no longer exuding.

What is claimed is:

1. A method for preventing infectious microorganisms from spreading from open sores or wounds comprising applying to said open sore or wound a quantity of a cyanoacrylate sufficient to cover and seal said open wound or sore.

2. The method of claim 1 wherein said open sore or wound results from the Herpes Simplex virus.

3. The method of claim 1 wherein a quantity of the cyanoacrylate applied is also sufficient to cover the peripheral skin area surrounding said open sore or wound.

4. The method of claim 1 wherein the amount of the cyanoacrylate applied is sufficient to form a film over said open sore or wound of from about 0.01 mm. to about 0.5 mm. thick.

5. The method of claim 4 wherein said film is from about 0.05 mm. to about 0.3 mm. thick.

6. The method of claim 3 wherein said cyanoacrylate is applied to extend outwardly and cover said peripheral skin area a distance of from about 0.05 cm. to about 0.5 cm. from said open sore or wound.

7. The method of claim 6 wherein said distance is from about 0.1 cm. to about 0.3 cm.

8. A method for preventing infectious microorganisms from spreading from open sores or wounds resulting from the Herpes Simples virus comprising applying directly to said open sore or wound a quantity of butyl-cyanoacrylate sufficient to cover and seal said open sore or wound.

9. The method of claim 8 wherein the quantity of said butyl-cyanoacrylate applied is also sufficient to cover the peripheral skin area surrounding said open sore or wound.

10. The method of claim 8 wherein the amount butyl-cyanoacrylate applied is sufficient to form a film over said open sore or wound of from about 0.01 mm. to about 0.5 mm. thick.

11. The method of claim 10 wherein said film is from about 0.05 mm. to about 0.3 mm. thick.

12. The method of claim 9 wherein said butyl-cyanoacrylate is applied to extend outwardly and cover said peripheral skin area a distance of from about 0.05 cm. to about 0.5 cm. from said open sore or wound.

13. The method of claim 12 wherein said distance is from about 0.1 cm. to about 0.3 cm.

14. A method for preventing infectious micro-organisms from spreading from open sores or wounds resulting from the Herpes Simplex virus comprising applying directly to said open sore or wound a quantity of octyl-cyanoacrylate sufficient to cover and seal said open sore or wound and form a film over said open sore or wound of from about 0.01 mm. to about 0.5 mm. thick.

15. The method of claim 14 wherein said film is from about 0.05 mm. to about 0.3 mm. thick.

16. The method of claim 14 wherein the quantity of said octyl-cyanoacrylate applied is also sufficient to cover the peripheral skin area surrounding said open sore or wound.

17. The method of claim 16 wherein said octyl-cyanoacrylate is applied to extend outwardly and cover said peripheral skin area a distance of from about 0.05 cm. to about 0.5 cm. from said open sore or wound.

18. The method of claim 17 wherein said distance is from about 0.1 cm. to about 0.3 cm.

19. The method of claim 1 wherein said cyanoacrylate is a member of the group consisting of butyl-cyanoacrylate, methyl-cyanoacrylate, allyl-cyanoacrylate, isobutyl-cyanoacrylate, ethyl-cyanoacrylate or octyl-cyanoacrylate.

20. The method of claim 2 wherein a quantity of ethyl-cyanoacrylate applied is also sufficient to cover the peripheral skin -area surrounding said open sore or wound.

21. The method of claim 2 wherein the amount of ethyl-cyanoacrylate applied is sufficient to form a film over said open sore or wound of from about 0.01 mm. to about 0.5 mm. thick.

22. The method of claim 2 wherein a quantity of 2 acetyl-cyanoacrylate applied is also sufficient to cover the peripheral skin area surrounding said open sore or wound.

23. The method of claim 2 wherein the amount of 2 acetyl-cyanoacrylate applied is sufficient to form a film over said open sore or wound of from about 0.01 mm. to about 0.5 mm. thick.

24. The method of claim 19 wherein the cyanoacrylate is n-butyl-cyanoacrylate.

25. The method of claim 19 wherein the cyanoacrylate is ethyl-cyanoacrylate.

26. The method of claim 19 wherein the cyanoacrylate is 2 acetyl-cyanoacrylate.

27. The method of claim 2 wherein the cyanoacrylate is n-butyl-cyanoacrylate.

28. The method of claim 2 wherein the cyanoacrylate is ethyl-cyanoacrylate.

29. The method of claim 2 wherein the cyanoacrylate is acetyl-cyanoacrylate.

30. The method of claim 2 wherein the cyanoacrylate is octyl-cyanoacrylate.
